(19) United States
(12) Patent Application Publication
(10) Pub. No.: US 2018/0014914 A1
(43) Pub. Date: Jan. 18, 2018

(54) SELF-ILLUMINATING CHEEK RETRACTOR

(71) Applicant: SREEVATSAN RAGHAVAN, Noida, OT (IN)

(72) Inventors: SREEVATSAN RAGHAVAN, NOIDA (IN); MANVENDRA SINGH GAHLOT, SIKAR (IN)

(21) Appl. No.: 15/544,659

(22) PCT Filed: Jan. 19, 2016
(86) PCT No.: PCT/IB2016/050244

§ 371 Date: Jul. 19, 2017

(30) Foreign Application Priority Data
Jan. 20, 2015 (IN) 172/DEL/2015

(51) Int. Cl. A61M 16/01; A61B 1/06; A61B 1/24; A61B 17/02; A61C 17/06; A61C 1/08; A61N 5/06; A61M 39/26; A61B 5/00; A61B 1/04; A61C 7/00; A61N 5/067; A61B 17/00

(52) U.S. Cl. CPC A61C 5/90; A61M 16/01; A61B 1/04; A61B 1/063; A61B 1/0646; A61B 1/24; A61B 17/0206; A61C 17/043; A61C 7/00; A61C 1/088; A61N 5/0601; A61M 39/26; A61B 5/0088; A61B 2090/304; A61B 2017/00557; A61B 2017/0212; A61M 2202/0283; A61B 2090/0813; A61N 2005/067; A61N 2005/0606

(57) ABSTRACT

A novel chemi-luminescent cheek retractor, wherein: the chemi-luminiscence is not localized to a specific location but the whole cheek retractor has multiple compartments to provide adequate and uniform illumination. Further, the cheek retractor is reversibly inflatable; chemi-luminiscence is triggered by the using the nitrous and ozone gas that pumped in to selective multiple compartments; a fiber reinforced latch and adjustable screw is provided to control unrestrained opening or closing, thereby minimizing the damage due to the uncontrolled elastic reflex of the material to the device and due to the force exerted on the jaws of the patient; the chambers also lined with a reflective coating on the inner surface to aid in better and uniform illumination; a multi-function valve is provided at the split junction capable of allowing various reversible attachments e.g. a suction tube, one LED diode-charged, wireless transmitter, camera etc.

### FIELD OF THE INVENTION

The present invention provides a novel self-illuminating cheek retractor. More particularly, the invention relates to an improved apparatus wherein the chemical luminescence is not compartmentalized and localized; however the entire cheek retractor (angular oris) including the handle(s) provide light from various angles and is directed by specialized coating, to eliminate shadowing while performing a procedure.

### BACKGROUND OF THE INVENTION

Dental operatory light (DOL) is one of the most important equipments that augment a dentist's ability to diagnose or treat patient's oral cavity. The pre requisites for good DOL are safety, energy efficiency, durability, heat management and hassle free mounting, positioning and cleaning. The conventional DOLs are designed keeping in mind that the dentist is close to the patient and shall carry various instruments therefore these lights are specifically designed to eliminate any possible shadowing that may occur in the mouth cavity during treatment. This has made it an almost universal practice for the dentist to employ movable light sources comparatively close to the patient. Therefore, DOL are prominently mounted for suitable reach and configuration.

Most of the times, it is also a desirable practice to swing the chair to some extent so that high intensity of illumination is incident onto the surfaces on which the dentist is working, so that operations are done under adequate lighting; all of which must be received through the comparatively small opening of the mouth.

The main drawback of these conventional DOL are that they are large and bulky are cluttered up for space, therefore constant adjustment is required; moreover constant adjustment of the dental chair is also required each time a patient moves. Such adjustments take the attention of the dentist away from the more important work in hand, are unsanitary, and annoying to the patient. All this is done to get the best incidence of the light. In addition to the above drawbacks, since the lights are bright and directed towards the face of a patient for obvious reasons, it is uncomfortable for a patient to keep eyes open during the proceedings.

Another major drawback of the DOL especially with reference to India is the ability to function in absence of basic essential commodity such as electricity. The problem is more prevalent in rural areas where most of the population resides. In absence of electricity, all these DOL technologies and other inventions for lighting oral cavity become irrelevant. An already available option is to set up power supply using a generator. This is a feasible at places where there are infrastructure to avail it, but where transient camps are installed; temporary arrangements are to be made for power supply.

In view of such circumstances the backup systems are economically not viable; moreover there is always a fear of over loading the system and starting a fire. Accordingly, there is a need of such devices which overcome the aforementioned shortcomings in the state of the art and provide a viable, economic and energy efficient solution for the same.

Although the use of chemi-luminance for illuminating the bucccal cavity is not new, however there are many limitation to the prior art.

U.S. Pat. No. 7,153,131, provides an intraoral illumination device for illuminating the interior of the mouth of a dental patient. The device comprises a flexible container having container defining walls, a portion of the walls being light transmitting. The device is sized for insertion into the mouth and placement against an interior surface of the mouth, e.g., the gum. A pressure-sensitive adhesive layer covers a portion of the walls for adhesively mounting the device to the interior surface of the mouth. A first chamber and a second chamber are within the container. The chambers are connected to each other by a frangible wall. Each of the chambers contain a chemical composition that when mixed together create a chemi-luminescent reaction that produces light. When the frangible wall is fractured by flexing the container, it permits the compositions to mix within the container to create a chemi-luminescent reaction that emits light through the container walls to illuminate the mouth, permitting dental procedures to be easily performed.

U.S. Pat. No. 6,299,441 B1, provides product includes a holder to be held inside the mouth of a user and a carrier to be held removably by the holder and having a first compartment containing a chemi-luminescent solution separated from a second compartment containing an activator for the chemi-luminescent solution by a frangible barrier. The carrier may be inserted into the holder and the holder flexed to break the barrier and mix the chemi-luminescent solution and activator to produce chemi-luminescent light inside the mouth of a user. The carrier can be replaced upon the light produced diminishing.

The problem with the prior art is that all of the inventions provide just one light source either in the centre or attached to the opposite end of the target, i.e. a device is placed with the help of an adhesive or a tubular strip inside the buccal cavity chemi-luminance illuminates the buccal cavity. However, as any surgeon shall specify that uniform and adequate DOL is the primary requirement i.e. there should be enough light that uniformly illuminates the buccal cavity from more than one source or direction, to eliminate the shadow formation while working. Placing a tube in certain designated area does not compensate for uniform lighting, further if such placement is done in the middle or directly opposite to the place to be examined there shall be shadowing.

Further as dental procedures require sufficient time, therefore a support should be provided to open the buccal cavity for extended period of time.

There are other problems associated such as storage, as these are of very unusual shape and a non-flexible material; therefore storage to avoid breakage is a vital issue.

The device should facilitate multiple actions and functions such as a valve at the split junction should be provided, making it capable to attach a suction tube to facilitate saliva ejection and at least one LED diode-charged externally by a capacitor, operated via a wireless transmitter to sync the flash of the camera during intra-oral photography.

The device should be easy to clean, should allow saliva and other fluids to drain properly, adequately and quickly, without taking out the support or the retractor. There should be provisions to provides a detachable window for intra-oral camera/laser diode/photo-spectrometer for detecting lesions like leukoplakia and oral lichen planus which are known to have different refractive properties when a chemi-luminiscent light is shined on such diseased tissue; and optionally a tongue guard with adhesive base chemi-luminscent pads should be provided to be attached on the undersurface of the tongue area for the dual purpose of isolation and illumination.

Other major problem that needs to be referred is the force exerted by the cheek retractor that exerts uncontrollable amount of force, to split the upper and lower jaw and maintain it in such position.

Therefore, there is a technological gap wherein a novel chemi-luminescent cheek retractor is required to overcome all the existing problems.

### OBJECT OF THE INVENTION

Accordingly, the main object of the invention is to provide a novel chemi-luminescent cheek retractor, that does not have compartmentalized and localized chemi-luminescence; however the entire cheek retractor (angular oris) including the handle(s) provides light from various angles and is directed by specialized coating to eliminate shadowing while performing a procedure.

Another object of the invention is to provide a method of chemi-luminescence in cheek retractor, wherein the method comprises allowing chemical reagents to make contact resulting in chemi-luminescence.

Yet another object of the invention is to provide a chemi-luminescent cheek retractor which is safe, reusable and easy to clean.

Yet another object of the invention is to produce the chemi-luminescent cheek retractor to provide adequate lighting with uniform intensity and high color rendering index (CRI).

Yet another object of the invention is to provide a chemi-luminescent cheek retractor tamperproof and light weight.

Yet another object of the invention is to provide a chemi-luminescent cheek retractor to produce at least 4 hours of sustainable light.

Yet another object of the invention is to provide a chemi-luminescent cheek retractor with replaceable/refillable chemical.

Yet another object of the invention is to provide a chemi-luminescent cheek retractor that is easily sterilized.

Yet another object of the invention is to provide a chemi-luminescent cheek retractor that produces different colors of light other than the standard yellow light that shall mimic the natural light.

Yet another object of the invention is to provide a chemi-luminescent cheek retractor that is reversibly inflatable, stored in deflated state and inflated for use, with nitrous oxide through a pressure valve thereby allowing the necessary conformational change required for affixing it.

Yet another object of the invention is to provide a chemi-luminescent cheek retractor that is used to provide the chemi-luminiscent reaction by placing an ozone trigger for the reaction.

Yet another object of the invention is to provide a chemi-luminescent cheek retractor that has at least one fiber reinforced latch and adjustable screw provided to control unrestrained opening or closing, allowing selective expansion or contraction of the retractor, thereby minimizing the damage due to the uncontrolled elastic reflex of the material to the device and due to the force exerted on the jaws of the patient.

Yet another object of the invention is to provide a chemi-luminescent cheek retractor that has the chambers housing the gas or the liquid providing the chemi-luminiscence reaction, are lined with a reflective coating on the inner surface to aid in better and uniform illumination.

Yet another object of the invention is to provide a chemi-luminescent cheek retractor that has a valve at the split junction capable of attaching a suction tube to facilitate saliva ejection and at least one LED diode-charged externally by a capacitor, operated via a wireless transmitter to sync the flash of the camera during intra-oral photography.

Yet another object of the invention is to provide a chemi-luminescent cheek retractor that has at least one detachable extendable flange(s) to the buccal mucosa that aid in isolation by providing a channel for removing saliva.

Yet another object of the invention is to provide a chemi-luminescent cheek retractor that has provides a closed channel for local anesthesia by utilizing dermal adhesive pads.

Yet another object of the invention is to provide a chemi-luminescent cheek retractor that has provides a detachable window for intra-oral camera/laser diode/photo-spectrometer for detecting lesions like leukoplakia and oral lichen planus which are known to have different refractive properties when a chemi-luminiscent light is shined on such diseased tissue.

Yet another object of the invention is to provide a chemi-luminescent cheek retractor that has the similar laser diode could be also used to provide phototherapy for treatment of lichen planus/leukoplakia and also to speed orthodontic treatment via photo-bio-modulation.

In yet another embodiment the cheek retractor has multi-detachable components to faciliate easy cleaning and sterilization of the retractor.

In yet another embodiment the cheek retractor optionally a tongue guard with adhesive base chemi-luminscent pads are provided to be attached on the undersurface of the tongue area for the dual purpose of isolation and illumination; and optionally nitrous oxide can be delivered through the retractor efficiently as an anesthetic.

In yet another object of the invention is to use nitrous oxide which is readily available with most dentists as it is a commonly used anesthetic hence additional chemical procurement is not necessary making it more cost effective.

### SUMMARY OF THE INVENTION

The present invention provides a novel self-illuminating cheek retractor. More particularly, the invention relates to an improved apparatus for Dental operatory light. The invention provides chemical luminescence within a cheek retractor for adequate lighting inside the oral cavity with uniform intensity and high color rendering index (CRI), without any external power source.

The present invention overcomes all drawbacks in state of the art technology, by providing a chemi-luminescent cheek retractor comprising a housing of transparent heat resistant flexible material such as silicone based material; a means for storage of chemical reagents in any form; and provisions for allowing chemi-luminescence on laser activation. The conventional cheek retractor housing only two localized troughs within for at least two chemical reagents and the light is generated when the retractor is flexed beyond a point to cause the reagents to mix with each other and the chemical reaction results in production of light. The present invention provides a gas filled continuous trough that is activated on LASER exposure, thus triggering chemi-luminescence.

In an embodiment of the current invention, the cheek retractor has continuous chamber housing the gas or the fluid, providing the chemi-luminiscent reaction. These chambers are lined with reflective coating to aid in better and uniform illumination, and directing the light towards the buccal cavity. The continuous chamber extends from angular oris to the handle(s). As the lighting is not secluded to one part, rather the lighting is generated from angular oris to handle which is away from the patients buccal cavity, therefore the internal coating ensures that the lighting is not wasted and is more focused towards the cavity. An extended lighting source also ensures that shadowing is minimized due to multiple sources of light and overlapping fields of illumination. The invention provides a novel cheek retractor, housing a lighting system that is LASER activated lighting system that does not require external power source and is economically viable. The cheek retractor lighting system provides at least 4 hours of luminance. The perishable chemical reagents, in any form, can be easily replaced so the device can be used incessantly after disinfection.

In yet another embodiment, the cheek retractor is reversibly inflatable, stored in deflated state and inflated for use, with nitrous oxide, wherein means for reversibly connecting a pressure valve is provided, thereby executing the necessary conformational change. The nitrous is used to provide chemi-luminiscence by an ozone trigger or a LASER trigger.

In yet another embodiment, the cheek retractor has at least one fiber reinforced latch and adjustable screw, provided to control unrestrained opening or closing, allowing selective expansion and/or contraction of the retractor, thereby minimizing the damage due to the uncontrolled elastic reflex the device, and due to the force exerted on the jaws of the patient.

In yet another embodiment the cheek retractor has at least one valve or similar means at the split junction capable of attaching a suction tube to facilitate saliva ejection and at least one LED diode-charged externally or a similar means to be operated via a wireless transmitter to sync the flash of the camera during intra-oral photography.

In yet another embodiment the cheek retractor has at least one detachable extendable flange or a similar means that a). aid in isolation by providing a channel for removing saliva; b). provides a closed channel for local anesthesia by utilizing dermal adhesive pads; c). provides a detachable window for intra-oral camera/laser diode/photo-spectrometer, for detecting lesions like leukoplakia and oral lichen planus, which are known to have different refractive properties when a chemiluminiscent light is shined on such diseased tissue; and at least one laser diode could be also used to provide phototherapy for treatment of lichen planus/leukoplakia and also to speed orthodontic treatment via photo-bio-modulation.

In yet another embodiment the cheek retractor has multi-detachable components to facilitate easy cleaning and sterilization of the retractor.

In yet another embodiment the cheek retractor is optionally a tongue guard with adhesive base chemi-luminscent pads, provided to be attached on the undersurface of the tongue area, for the dual purpose of isolation and illumination; and optionally nitrous oxide can be delivered through the retractor efficiently as an anesthetic.

### BRIEF DESCRIPTION OF THE DRAWINGS

A complete understanding of the system and method of the present invention may be obtained by reference to the following drawings:

FIG. 1 elucidates the preferred embodiment of the novel chemi-luminescent cheek retractor from the top view;

FIG. 2 elucidates the preferred embodiment of the novel chemi-luminescent cheek retractor from the top perspective view; and

FIG. 3 elucidates the preferred embodiment of the novel chemi-luminescent cheek retractor from the front perspective view.

### DETAILED DESCRIPTION OF THE INVENTION

The present invention will now be described more fully hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention provides a novel self-illuminating cheek retractor. More particularly, the invention relates to an improved apparatus for dental illumination. The invention provides chemical luminescence within a cheek retractor for adequate lighting inside the oral cavity with uniform intensity and high color rendering index (CRI).

In an embodiment of the current invention, the cheek retractor has continuous chamber housing the gas or the fluid, providing the chemi-luminiscent reaction. These chambers are lined with reflective coating to aid in better and uniform illumination, and directing the light towards the buccal cavity. The continuous chamber extends from angular oris to the handle(s). As the lighting is not secluded to one part, rather the lighting is generated from angular oris to handle which is away from the patients buccal cavity, therefore the internal coating ensures that the lighting is not wasted and is more focused towards the cavity. An extended lighting source also ensures that shadowing is minimized due to multiple sources of light and overlapping fields of illumination. The invention provides a novel cheek retractor, housing a lighting system that is LASER activated lighting system that does not require external power source and is economically viable. The cheek retractor lighting system provides at least 4 hours of luminance. The perishable chemical reagents, in any form, can be easily replaced so the device can be used incessantly after disinfection.

The cheek retractor is tamperproof and light weight. The transparent cover or the chemical reagents can be varied to emit different spectrum of light other than the standard yellow issue that mimics the natural light. The teeth retractor may be metallic, non metallic or polymer which are inert to the chemical reagents and reaction.

In an embodiment of the current invention a novel chemi-luminescent cheek retractor, comprising a housing of transparent heat resistant flexible material such as silicone based materials, means for storage of chemical reagents, provision for allowing contact between the separated chemical reagents to produce chemi-luminescence, chemi-luminiscence is triggered by using the nitrous and ozone gas; wherein: the cheek retractor is reversibly inflatable, stored in deflated state and inflated for use, with gasses such as nitrous oxide through a pressure valve thereby allowing the necessary conformational change required for affixing it.

The invention provides a novel cheek retractor, housing a lighting system LASER activated lighting system that does not require external power source and is economically viable. The cheek retractor lighting system provides at least 4 hours of luminance. The chemi-luminescent cheek retractor is affixed into the oral cavity of the patient therefore there are no adjustments or hindrances or shadows. The perishable chemical reagents, in any form, can be easily replaced so the device can be used incessantly after disinfection.

In yet another embodiment the cheek retractor is reversibly inflatable, stored in deflated state and inflated for use, with nitrous oxide wherein means for reversibly connecting a pressure valve is provided, thereby executing the necessary conformational change. The nitrous is used to provide the chemi-luminiscence by an ozone trigger or a LASER trigger.

In yet another embodiment, the cheek retractor has at least one fiber reinforced latch and adjustable screw, provided to control unrestrained opening or closing, allowing selective expansion and/or contraction of the retractor, thereby minimizing the damage due to the uncontrolled elastic reflex the device, and due to the force exerted on the jaws of the patient.

In yet another embodiment the cheek retractor has the chambers housing the gas or the liquid providing the chemi-luminiscent reaction are lined with a reflective coating on the inner surface to aid in better and uniform illumination, and directing the light towards the buccal cavity. As the lighting is not secluded to one part, rather the lighting is generated from the handle which is away from the patients buccal cavity, therefore the lining ensures that the lighting is not wasted and is more focused towards the cavity. This also ensures that shadowing is minimized due to multiple sources of light and overlapping fields of illumination.

In yet another embodiment the cheek retractor has at least one valve or similar means at the split junction capable of attaching a suction tube to facilitate saliva ejection and at least one LED diode-charged externally or a similar means to be operated via a wireless transmitter to sync the flash of the camera during intra-oral photography.

In yet another embodiment the cheek retractor has at least one detachable extendable flanges to the buccal mucosa or a similar means that a). aid in isolation by providing a channel for removing saliva; b). provides a closed channel for local anaesthesia by utilizing dermal adhesive pads; c). provides a detachable window for intra-oral camera/laser diode/photo-spectrometer, for detecting lesions like leukoplakia and oral lichen planus, which are known to have different refractive properties when a chemi-luminiscent light is shined on such diseased tissue; and at least one laser diode could be also used to provide phototherapy for treatment of lichen planus/leukoplakia and also to speed orthodontic treatment via photo-bio-modulation.

In yet another embodiment the cheek retractor has multi-detachable components to faciliate easy cleaning and sterlization of the retractor.

In yet another embodiment the cheek retractor is optionally a tongue guard with adhesive base chemi-luminscent pads, provided to be attached on the undersurface of the tongue area, for the dual purpose of isolation and illumination; and optionally nitrous oxide can be delivered through the retractor efficiently as an anesthetic.

In an embodiment of the current invention a novel chemi-luminescent cheek retractor, comprising a housing of transparent heat resistant flexible material such as silicone based materials, means for storage of chemical reagents, provision for allowing contact between the separated chemical reagents to produce chemi-luminescence, chemi-luminiscence is triggered by using the nitrous and ozone gas; wherein: a valve at the split junction is provided capable of attaching reversibly attachable components, for providing multi-functionality, the purpose of detachable components is to facilitate easy cleaning and sterilization, at least one detachable extendable flanges to the buccal mucosa that:

a. aid in isolation by providing a channel for removing saliva;
b. provides a closed channel for local anaesthesia by utilizing dermal adhesive pads; and
c. provides a detachable window for intra-oral camera/laser diode/photo-spectrometer for detecting lesions like leukoplakia and oral lichen planus which are known to have different refractive properties when a chemi-luminiscent light is shined on such diseased tissue and at least one laser diode could be also used to provide phototherapy for treatment of lichen planus/leukoplakia and also to speed orthodontic treatment via photo-bio-modulation.

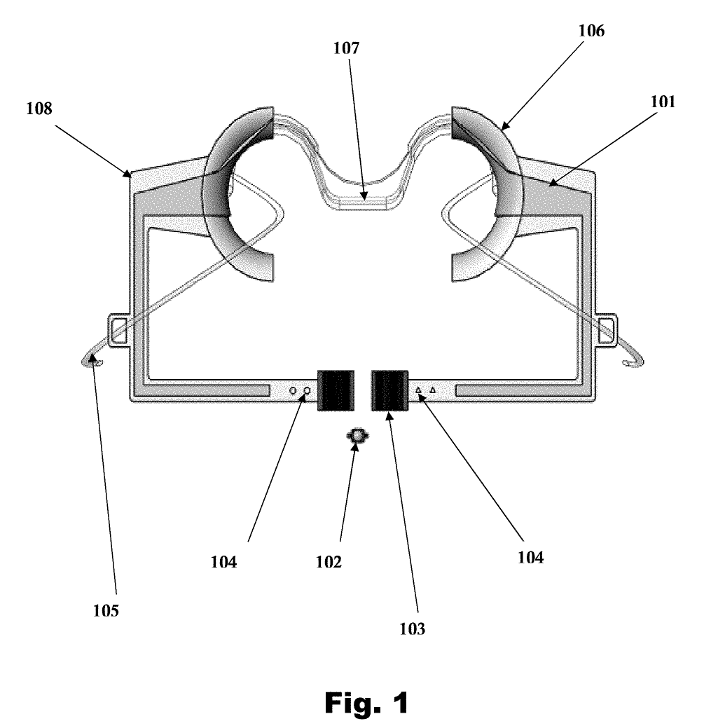

FIG. 1, elucidates the preferred embodiment of the novel chemi-luminescent cheek retractor wherein Chemi-luminescent chamber 101 is not localized to one side, instead it runs through the handle 108 and angular oris 106. Further, Chemi-luminescent chamber 101 are lined with reflective coating to focus the light inside the buccal cavity, as Chemi-luminescent chamber 101 extends from angular oris 106 to the handle 108, therefore the reflecting coating is selectively applied to focus the light towards buccal cavity and minimizing shadowing, by overlapping the field of illumination. The attachment knob 102 allows the gadgets or any attachments inserted into the fastening groove 103 to be secured properly, making it more versatile. Multiple attachments are possible or even adjustments can be made due to, extra attachment slots 104. Further, at least one detachable extendable flanges are provided 105 to abet attachments for aid in isolation by providing a channel for removing saliva; provides a closed channel for local anaesthesia by utilizing dermal adhesive pads; and provides a detachable window for intra-oral camera/laser diode/photo-spectrometer for detecting lesions like leukoplakia and oral lichen planus which are known to have different refractive properties when a chemi-luminiscent light is shined on such diseased tissue and at least one laser diode could be also used to provide phototherapy for treatment of lichen planus/leukoplakia and also to speed orthodontic treatment via photo-bio-modulation.

At least one tongue away shield 107 is provided that folds or keeps the tongue away during procedures and therefore, minimizes accidents.

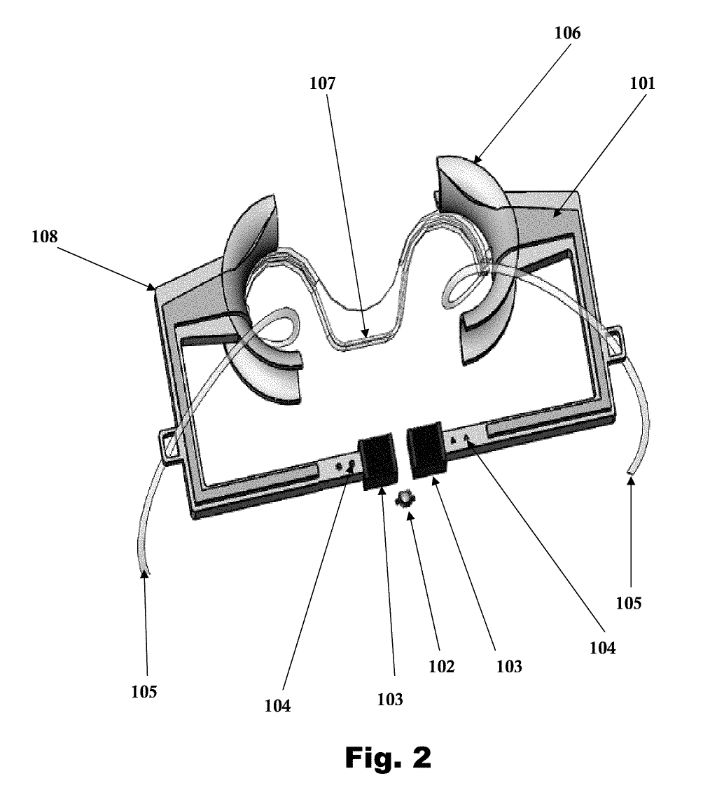

Further illustration of the preferred embodiment is shown in FIG. 2 elucidates the preferred embodiment of the novel chemi-luminescent cheek retractor from the top perspective view; and

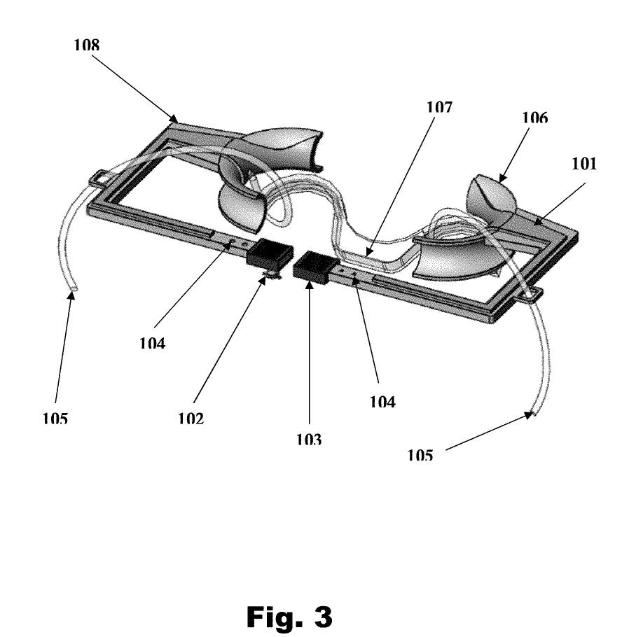

FIG. 3 elucidates the preferred embodiment of the novel chemi-luminescent cheek retractor from the front perspective view.

While the best mode has been described in detail, those familiar with the art will recognize various alternative designs and examples within the scope of the following claims.

I claim:

1. A novel chemi-luminescent cheek retractor comprising a housing of transparent heat resistant flexible material, means for storage of chemical reagents, provision for allowing contact between the separated chemical reagents to produce chemi-luminescence triggered by using the nitrous and ozone gas; wherein:
a) the cheek retractor is reversibly inflatable, stored in deflated state and inflated for use, with gas such as nitrous oxide through a pressure valve thereby allowing the necessary conformational change required for affixing it;
b) a fiber reinforced latch and adjustable screw is provided, to control unrestrained opening or closing, allowing selective expansion or contraction of the retractor, thereby minimizing the damage due to the uncontrolled elastic reflex of the material to the device and due to the force exerted on the jaws of the patient;
c) the chambers housing the gas or the liquid allowing the chemi-luminiscent reaction lined with a reflective coating on the inner surface to aid in better and uniform illumination;
d) a valve at the split junction is provided capable of attaching reversibly attachable components, for providing multi-functionality, the purpose of detachable components is to facilitate easy cleaning and sterilization;
e) at least one detachable extendable flanges to the buccal mucosa; and
optionally a tongue guard with adhesive base chemi-luminscent pads are provided to be attached on the undersurface of the tongue area for the dual purpose of isolation and illumination; and
optionally nitrous oxide can be delivered through the retractor efficiently as an anesthetic.

2. The device as claimed in claim 1, wherein the heat resistant flexible material shall include silicone based material.

3. The device as claimed in claim 1, wherein the chemical reagents are stored in any form such as solid, liquid, gas or any combination thereof.

4. The device as claimed in claim 1, wherein the transparent casing over the chemi-luminescent cheek retractor can be supplemented with filters of different wavelength.

5. The device as claimed in claim 1, wherein at least one detachable extendable flanges to the buccal mucosa aid in isolation by providing a channel for removing saliva.

6. The device as claimed in claim 1, wherein at least one detachable extendable flanges provides a closed channel for local anaesthesia by utilizing dermal adhesive pads.

7. The device as claimed in claim 1, wherein valve split junction shall accommodate at least one suction tube to facilitate saliva ejection and at least one LED diode-charged externally by a capacitor, operated via a wireless transmitter to sync the flash of the camera during intra-oral photography;

8. The device as claimed in claim 1, wherein at least one detachable extendable flanges provides a detachable window for intra-oral camera/laser diode/photo-spectrometer for detecting lesions like leukoplakia and oral lichen planus which are known to have different refractive properties when a chemi-luminiscent light is shined on such diseased tissue.

9. The device as claimed in claim 1, wherein at least one detachable extendable flanges the similar laser diode could be also used to provide phototherapy for treatment of lichen planus/leukoplakia and also to speed orthodontic treatment via photo-bio-modulation.

10. The device as claimed in claim 1, wherein chemi-luminesce is triggered by using the nitrous and ozone gas through a laser beam.
